(19) United States
(12) Patent Application Publication
(10) Pub. No.: US 2023/0121797 A1
(43) Pub. Date: Apr. 20, 2023

(54) A COMBINATION OF FLAVONOIDS AND SPHINGOSINE 1 PHOSPHATE LYASE INHIBITORS FOR THE TREATMENT OF LUNG INFLAMMATION

(71) Applicant: AC BIOSCIENCE SA, Epalinges (CH)

(72) Inventors: Christian AUCLAIR, Saint-Arnoult En Yvelines (FR); Annette IVES, Ecublens (CH)

(21) Appl. No.: 17/914,469

(22) PCT Filed: Mar. 25, 2021
(86) PCT No.: PCT/EP2021/057701

§ 371 Date: Sep. 26, 2022

(30) Foreign Application Priority Data
Mar. 27, 2020 (EP) 20166150.1

(51) Int. Cl. A61K 31/417; A61P 11/00; A61P 29/00; A61K 31/353

(52) U.S. Cl. CPC A61K 31/417; A61P 11/00; A61P 29/00; A61K 31/353

(57) ABSTRACT

The invention provides a pharmaceutical combination of a flavonoid compound and a sphingosine-1-phosphate lyase inhibitor (S1PLI) for simultaneous, separate or sequential administration, and use thereof for ameliorating and/or reducing the lung inflammation, resulting from MERS-CoV and/or SARS-CoV virus infection, during and following the virus infection.

### FIELD OF THE INVENTION

The invention relates to a pharmaceutical combination of a flavonoid compound and a sphingosine-1-phosphate lyase inhibitor (S1PLI) for simultaneous, separate or sequential administration, and use thereof for ameliorating and/or reducing the lung inflammation, resulting from MERS-CoV and/or SARS-CoV virus infection, during and following the virus infection.

### BACKGROUND OF THE INVENTION

Lung Pathogenesis Associated to SARS-CoV Infection

COVID-19 is a respiratory disease caused by a coronavirus (SARS-CoV-2) and causes substantial morbidity and mortality. There is currently neither vaccine to prevent Covid-19 or infection with SARS-CoV-2 nor therapeutic agent to treat COVID-19. Coronavirus (CoVs) are positive-sense single stranded enveloped RNA viruses, many of which are commonly found in humans and cause mild symptoms. Over the past two decades, emerging pathogenic CoVs capable of causing life-threatening disease in humans and animals have been identified, namely severe acute respiratory syndrome coronavirus (SARS-CoV) and Middle Eastern respiratory syndrome coronavirus (MERS-CoV). This novel coronavirus has been designated SARS-CoV-2, and the disease caused by this virus has been designated COVID-19. There is currently no treatment approved in the treatment of patients with COVID-19. COVID-19 infection causes clusters of severe respiratory illness similar to severe acute respiratory syndrome coronavirus (SARS-CoV) and MERS-CoV and is associated with intensive care unit admission and high mortality (Xu et al. 2020). COVID-19 pneumonia manifests with chest computed Tomography (CT) imaging abnormalities, even in asymptomatic patients. On hospital admission, abnormalities in chest CT images were detected among all patient (Shi et al. 2020). Complications included acute respiratory distress syndrome (29% cases) (Xu et al. 2020), acute cardiac injury (12%) and secondary infection (10%).

Histopathological observations and imaging features of pulmonary lesions in COVID-19 patients overlap with those of SARS-CoV and MERS-CoV (Hosseini et al. 2020). COVID-2019 patients present non-specific inflammatory responses, including oedema and inflammatory cell infiltration, and exhibit severe exfoliation of alveolar epithelial cells, alveolar septal widening, damage to alveolar septa, and alveolar space infiltration in a distinctly organized manner. This pathological inflammation includes tissue necrosis, infiltration, and hyperplasia. Thus, damage to the pulmonary interstitial arteriolar walls indicates that inflammatory response plays an important role throughout the course of disease in spite of the pathogenic effect in CoVs. These deleterious excessive and aberrant non-effective host immune responses are related to a “cytokine storm” (Li et al. 2020, Channappanavar et al. 2017) characterized by an increased plasma concentration of a number of pro-inflammatory cytokines and chemokines such as IL-1β, IL-1Rα, IL-2, IL-6, IL-7, IL-8, IL-9, IL17 and TNFα. Coupled with this are the observations that patients with severe COVID-19 infection in Wuhan, China displayed high leukocyte and PMN levels, reduced lymphocyte counts and high plasmatic inflammatory biomarkers (Qin et al, 2020, Zheng et al, 2020).

The Lung Inflammatory Process Following Viral Infection

The innate and adaptive immune systems play pivotal roles in orchestrating the host response to infection and tissue injury. The host responses to such insults include the production of a variety of pro-inflammatory cytokines and chemokines. In normal conditions, the induction of the pro-inflammatory response triggers the development of a counter-regulatory anti-inflammatory cytokine response to control inflammation and prevent excessive injury. In certain instances (e.g. infection with the highly pathogenic avian H5N1 or 1918 pandemic influenza virus strains and likely with the SARS-CoV), this counter regulation fails since infection results in a massive inflammatory cell infiltration into the infected lungs and excessive pro-inflammatory cytokine production.

The state of research clearly indicates that cells of the innate immunity including neutrophils, macrophages and mast cells, all together, play a major role during coronavirus infection. Neutrophils display properties leading to the hypothesis that these cells may act as a major driver of the inflammatory processes. Accordingly, neutrophil infiltration is constantly observed and sever lung inflammation. Experimental data reveal the actual occurrence of a chemokine dependent reciprocal crosstalk between neutrophils and Th17 cells, which may represent a major mechanism involved in the development of various inflammatory diseases. Along this line, neutrophils are a major player in tissue inflammation can efficiently produce cytokines including IL-17, TNF, IL-6, IL-8, and the chemokines such as CCL5 and CXL10, which propagates cellular influx. In addition to neutrophils, myeloid cells including macrophages, inflammatory monocytes and dendritic cells also play a role in inflammatory responses, viral clearance, and in adaptive immune responses during viral infection.

In terms of viral persistence SARS-CoV and MERS-CoV, as well as other + ssRNA viruses, are contained in double-membraned vesicles, which prevents the recognition of the ssRNA genome and generate immunosuppressive viral proteins (Jamieson, 2016 and Chen et al 2014). These factors can modulate the elicitation, and the temporal kinetics, of anti-viral type I IFN responses. In a murine model of SARs-CoV, delayed, but prolonged, type I IFN production by plasmacytoid Dcs in the lungs was directly related to the recruitment of innate immune mediators (including inflammatory monocytes-macrophages) and their activation (Channappanavar et al, 2016). These Type I IFNs subsequently induce the production of other pro-inflammatory cytokines and chemokines during lung infections, including IL-6 and TNF and are regulated by reactive oxygen species generation.

Among inflammatory cytokines, IL-6 seems to play a major role in the propagation of the inflammatory process. IL6 is produced by multiple cell types including fibroblasts, keratinocytes, mesangial cells, vascular endothelial cells, neutrophils, mast cells, macrophages, dendritic cells, and T and B cells in response to tissue damage and infections (Mauer et al. 2015). IL-6 facilitates the transition from the innate to adaptive immune response by promoting the recruitment, differentiation, and activity of monocytes and T cells. It is well known that dysregulated continual synthesis of IL-6 plays a pathological effect on chronic inflammation and autoimmunity.

Attention should be focused on neutrophils which have been implicated in the pathogenesis of many inflammatory lung diseases, including the acute respiratory distress syndrome, chronic obstructive pulmonary disease and asthma. IL-8 is a potent neutrophil recruiting and activating factor and the detection of IL-8 in clinical samples from patients with these diseases has led clinicians to believe that antagonism of IL-8 may be a practicable therapeutic strategy for disease management (Allen et al. 2014). Whilst lipopolysaccharide, IL-1beta and tumor necrosis factor-alpha are capable of augmenting IL-8 production. Regulation of the IL-8 gene is under the control of nuclear factor NFkB which appears to be a primary target for corticosteroid-mediated repression of IL-8 production. More specifically, it should be noticed that in the COVID-19 infected patients the loss of angiotensin converting enzyme 2 (ACE2) function can be a initiating event that leads to increased neutrophil infiltration in the lung and results in exaggerated inflammation and injury, as it was observed in disease models (Chinder et al. 2018).

The innate immunity response is followed by the trigger of adaptive immunity (Jones 2005) resulting in the infiltration and activation of CD4 and CD8 T lymphocytes. Activated antigen-specific T cells produce a variety of effector molecules contributing significantly to inflammation and tissue injury. Activated CD8+ T cells produce IFN-gamma-inducible protein-10 (CXCL10), induce production of macrophage elastase (matrix metalloproteinase 12) that degrades elastin, both causing lung destruction directly and generating elastin fragments that serve as monocyte chemokines augmenting macrophage-mediated lung destruction.

Substantial evidence suggests that CD8+ T cells contribute to the pathogenesis of a variety of lung diseases (Connors et al. 2016) such as chronic obstructive pulmonary disease (COPD) and lymphoid interstitial pneumonia (LIP). This suggestion is based in part on the observation that these disorders are characterized by the preferential accumulation of CD8+ T cells in the alveolar space of the interstitial tissue. Moreover, an effector cytokine profile has been detected in CD8+ T cells isolated from patients with pulmonary disorders; the presence of these cytokines suggests that T cytotoxic type 1 (Tc1) cells make an important contribution to the pathologic findings associated with lung diseases.

Morbidity is Directly Linked to the Extent of Lung Inflammation

Pathological examinations of samples obtained from patients who died of SARS revealed diffuse alveolar damage and morphological changes at various stages and of various degrees of severity, accompanied by prominent hyperplasia of pulmonary epithelial cells and presentation of activated alveolar and interstitial macrophages. Strikingly, these pulmonary manifestations were usually found after clearance of viremia and in the absence of other opportunistic infections, findings which suggest that intense local inflammatory responses could be responsible for the profound pulmonary pathology. The likelihood that SARS stems from excessive and uncontrolled inflammatory responses is supported by the detection of the reactive hemophagocytic syndrome, a disease caused by cytokine dysregulation, in the lungs of severely affected patients. Host-directed therapy could constitute a strategy of choice to efficiently treat COVID-19 patients by controlling inflammation.

Therefore, there is still a need for a treatment that can efficiently treat lung inflammation, resulting from MERS-CoV and/or SARS-CoV, during and following the viral infection (coronavirus infection).

### SUMMARY OF THE INVENTION

An aspect of the present invention provides a pharmaceutical combination of a flavonoid compound and a sphingosine-1-phosphate lyase inhibitor (S1PLI) for simultaneous, separate or sequential administration,
wherein the flavonoid compound is selected from the group comprising dihydroquercetin (DHQ), quercetin, astilbin, dihydrokaempferol, butin, eriodictyol, hesperetin, hesperidin, homoeriodictyol, isosakuranetin, naringenin, naringin, pinocembrin, poncirin, sakuranetin, sakuranin, sterubin, epigallocatechin gallate, catechin, epicatechin; preferably the flavonoid compound is selected from the group comprising dihydroquercetin (DHQ), quercetin, astilbin, epigallocatechin gallate, catechin and epicatechin; more preferably the flavonoid compound is selected from the group comprising dihydroquercetin (DHQ), quercetin, astilbin and epigallocatechin gallate;
wherein the sphingosine-1-phosphate lyase inhibitor (S1PLI) is a compound of Formula I

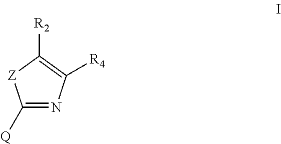

wherein
Z is selected from the group comprising O, S, NH, preferably Z is O or S or preferably Z is NH;
Q is

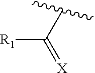

or optionally substituted heterocycle; preferably Q is

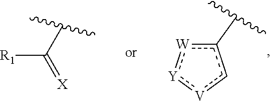

more preferably Q is X

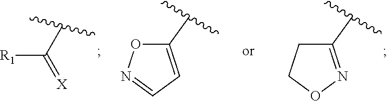

X is O or NR₃; preferably X is NR₃;
each of W, Y, V is independently selected from the group comprising CH₂, CH, N, NH, O or S;
R₁ is selected from the group comprising OR_{A}, NHOH, hydrogen, optionally substituted alkyl, optionally substituted aryl, optionally substituted alkylaryl, optionally substituted arylalkyl, optionally substituted heteroalkyl, optionally substituted heterocycle, optionally substituted alkylheterocycle, optionally substituted heterocyclealkyl; preferably R₁ is C₁-C₅ alkyl; more preferably R₁ is —CH₃;
R₂ is selected from the group comprising OR_{B}, C(O)OR_{B}, hydrogen, halogen, nitrile, optionally substituted hydroxyalkyl, optionally substituted alkyl, optionally substituted aryl, optionally substituted alkylaryl, optionally substituted arylalkyl, optionally substituted heteroalkyl, optionally substituted heterocycle, optionally substituted alkylheterocycle, optionally substituted heterocyclealkyl; preferably, R₂ is hydrogen or —(CH₂)ₙ—OH, wherein n is 1 to 5, preferably n is 1;
R₃ is selected from the group comprising OR_{C}, N(R_{C})₂, NHC(O)R_{C}, NHSO₂R_{C}, hydrogen; preferably, R₃ is —OH;
R₄ is selected from the group comprising OR_{D}, OC(O)R_{D}, N(R_{E})₂, hydrogen, halogen, optionally substituted hydroxyalkyl, optionally substituted alkyl, optionally substituted aryl, optionally substituted alkylaryl, optionally substituted arylalkyl, optionally substituted heteroalkyl, optionally substituted heterocycle, optionally substituted alkylheterocycle, optionally substituted heterocyclealkyl; preferably R₄ is hydrogen, —(CH₂)ₙ—OH, wherein n is 1 to 5, preferably n is 1 or C₄ hydroxyalkyl (such as tetrahydroxybutyl);
each of R_{A}, R_{B}, R_{C}, R_{D}, and R_{E} is independently selected from the group comprising hydrogen, optionally substituted alkyl, optionally substituted aryl, optionally substituted alkylaryl, optionally substituted arylalkyl, optionally substituted heteroalkyl, optionally substituted heterocycle, optionally substituted alkylheterocycle, or optionally substituted heterocyclealkyl.

Another aspect of the present invention provides a pharmaceutical combination of the invention for use in a method for ameliorating and/or reducing the lung inflammation, resulting from MERS-CoV and/or SARS-CoV virus infection, during and following the virus infection of a subject.

Another aspect of the present invention provides a sphingosine-1-phosphate lyase inhibitor for use in a method for ameliorating and/or reducing the lung inflammation, resulting from MERS-CoV and/or SARS-CoV virus infection, during and following the virus infection of a subject, the method comprising co-administering to the subject in need thereof a therapeutically effective amounts of a flavonoid compound and a sphingosine-1-phosphate lyase inhibitor.

### BRIEF DESCRIPTION OF THE FIGURES

FIG. 1 shows inhibitory effect of DHQ on the cytochrome c reduction rate (superoxide Anion production) resulting from the neutrophils activation by PMA or FLMP. Human neutrophils (2×10⁶/mL) were incubated in HBSS with TNF-α at 37° C. for 25 minutes. Cells were stimulated with FMLP or PMA in the presence of cytochrome c (1.2 mg/mL final), and the optical density of the supernatants was determined by spectrophotometry (550 nm). For both PMA or FLMP as PMN activators, the IC50 for DHQ inhibition is around 8 μM.

FIG. 2 shows inhibitory effect of DHQ on the ROS production resulting from the neutrophils activation by 10-6 M FMLP (A) or 100 n/ml PMA (B). ROS production is measured using a chemiluminescence standard procedure: briefly, One hundred microliters of human neutrophils (4×10⁶/mL) were primed with TNF-α at 37° C. for 25 minutes. 100 μL luminol (1 μM final concentration) and HRP (62.5 U/mL final concentration) in HBSS were added, and 150-μL aliquots were transferred to a prewarmed 96-well luminometer plate. Light emission was recorded by a Berthold MicroLumat Plus luminometer (Berthold Technologies, Hartfordshire, United Kingdom) (data output is in relative light units per second).

FIG. 3 shows degradation of S1P into phosphoethanolamine and hexadecenal as catalyzed by S1P lyase

FIG. 4 shows compound 2 treatment induces up to 60% depletion of circulating lymphocytes. Mice were treated with the indicated single oral doses of 2, and blood lymphocyte counts were measured 18 h after dosing. Data were pooled from three independent experiments, giving similar results and represent 8-10 mice each cohort. Data are presented as mean (SEM; *p<0.05) Bagdanoff et al. 2010).

FIG. 5 shows effect of LX2931 treatment on the circulating lymphocytes count. Data from phase Ia clinical trial: lymphocyte counts are reduced after treatment with compound LX2931 (2) with recovery 48 h after nadir. (A) Dose-dependent reduction in lymphocyte counts observed after single dose phase 1a trial: A total of 159 patients participated in a randomised, double-blinded, placebo-controlled, single and multiple ascending dose studies. Patient numbers were 120 and 39 for active dosing regimens and placebo regimens, respectively. (B) Rapid reduction of blood lymphocytes from a single 125 mg dose (N=6 patients) followed by a return to baseline in 48 h after nadir. (Bagdanoff et al., 2010). LX2931 was identified as “2” in A.

FIG. 6 shows simplified representation of the method of treatment according to the invention.

### DETAILED DESCRIPTION OF THE INVENTION

All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise” is generally used in the sense of include, that is to say permitting the presence of one or more features or components. Also as used in the specification and claims, the language “comprising” can include analogous embodiments described in terms of “consisting of” and/or “consisting essentially of”.

As used in the specification and claims, the singular form “a”, “an” and “the” include plural references unless the context clearly dictates otherwise.

As used in the specification and claims, the term “and/or” used in a phrase such as “A and/or B” herein is intended to include “A and B”, “A or B”, “A”, and “B”.

The term “alkyl” means a straight chain, branched and/or cyclic (“cycloalkyl”) hydrocarbon having from 1 to 20 or 1 to 10 or 1 to 4 carbon atoms. Alkyl moieties having from 1 to 4 carbons are referred to as “lower alkyl”. Examples of alkyl groups include, but are not limited to, methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl and dodecyl. Cycloalkyl moieties may be monocyclic or multicyclic, and examples include cyclopropyl, cyclobutyl, cyclopentyl, cyclohexyl, and adamantyl. Additional examples of alkyl moieties have linear, branched and/or cyclic portions (e.g., 1-ethyl-4-methyl-cyclohexyl). The term “alkyl” includes saturated hydrocarbons as well as alkenyl and alkynyl moieties.

The term “alkenyl” means a straight chain, branched and/or cyclic hydrocarbon having from 2 to 20 (e.g., 2 to 10 or 2 to 6) carbon atoms, and including at least one carbon-carbon double bond. Representative alkenyl moieties include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl and 3-decenyl.

The term “alkylaryl” or “alkyl-aryl” means an alkyl moiety bound to an aryl moiety.

The term “alkylheteroaryl” or “alkyl-heteroaryl” means an alkyl moiety bound to a heteroaryl moiety.

The term “alkylheterocycle” or “alkyl-heterocycle” means an alkyl moiety bound to a heterocycle moiety.

The term “alkynyl” means a straight chain, branched or cyclic hydrocarbon having from 2 to 20 or 2 to 20 or 2 to 6 carbon atoms, and including at least one carbon-carbon triple bond. Representative alkynyl moieties include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl and 9-decynyl.

The term “alkoxy” means an —O-alkyl group. Examples of alkoxy groups include, but are not limited to, —OCH₃, —OCH₂CH₃, —O(CH₂)₂CH₃, —O(CH₂)₃CH₃, —O(CH₂)₄CH₃, and —O(CH₂)₅CH₃.

The term “aryl” means an aromatic ring or an aromatic or partially aromatic ring system composed of carbon and hydrogen atoms. An aryl moiety may comprise multiple rings bound or fused together. Examples of aryl moieties include, but are not limited to, anthracenyl, azulenyl, biphenyl, fluorenyl, indan, indenyl, naphthyl, phenanthrenyl, phenyl, 1,2,3,4-tetrahydro-naphthalene, and tolyl.

The term “arylalkyl” or “aryl-alkyl” means an aryl moiety bound to an alkyl moiety.

The terms “halogen” and “halo” encompass fluorine, chlorine, bromine, and iodine.

The term “heteroalkyl” refers to an alkyl moiety (linear, branched or cyclic) in which at least one of its carbon atoms has been replaced with a heteroatom (such as N, O or S).

The term “heteroaryl” means an aryl moiety wherein at least one of its carbon atoms has been replaced with a heteroatom (such as N, O or S). Examples include, but are not limited to, acridinyl, benzimidazolyl, benzofuranyl, benzoisothiazolyl, benzoisoxazolyl, benzoquinazolinyl, benzothiazolyl, benzoxazolyl, furyl, imidazolyl, indolyl, isothiazolyl, isoxazolyl, oxadiazolyl, oxazolyl, phthalazinyl, pyrazinyl, pyrazolyl, pyridazinyl, pyridyl, pyrimidinyl, pyrimidyl, pyrrolyl, quinazolinyl, quinolinyl, tetrazolyl, thiazolyl, and triazinyl.

The term “heteroarylalkyl” or “heteroaryl-alkyl” means a heteroaryl moiety bound to an alkyl moiety.

The term “heterocycle” refers to an aromatic, partially aromatic or non-aromatic monocyclic or polycyclic ring or ring system comprised of carbon, hydrogen and at least one heteroatom (e.g., N, O or S). A heterocycle may comprise multiple (i.e., two or more) rings fused or bound together. Heterocycles include heteroaryls. Examples include, but are not limited to, benzo[1,3]dioxolyl, 2,3-dihydro-benzo[1,4]dioxinyl, cinnolinyl, furanyl, hydantoinyl, morpholinyl, oxetanyl, oxiranyl, piperazinyl, piperidinyl, pyrrolidinonyl, pyrrolidinyl, tetrahydrofuranyl, tetrahydropyranyl, tetrahydropyridinyl, tetrahydropyrimidinyl, tetrahydrothiophenyl, tetrahydrothiopyranyl and valerolactamyl.

The term “heterocyclealkyl” or “heterocycle-alkyl” refers to a heterocycle moiety bound to an alkyl moiety.

The term “heterocycloalkyl” refers to a non-aromatic heterocycle.

The term “heterocycloalkylalkyl” or “heterocycloalkyl-alkyl” refers to a heterocycloalkyl moiety bound to an alkyl moiety.

The term “substituted,” when used to describe a chemical structure or moiety, refers to a derivative of that structure or moiety wherein one or more of its hydrogen atoms is substituted with a chemical moiety or functional group such as, but not limited to, alcohol, aldehyde, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amidinyl (—C(NH)NH-alkyl or —C(NR)NH2), amine (primary, secondary and tertiary such as alkylamino, arylamino, arylalkylamino), aroyl, aryl, aryloxy, azo, carbamoyl (—NHC(O)O-alkyl- or —OC(O)NH-alkyl), carbamyl (for example CONH₂, as well as CONH-alkyl, CONH-aryl, and CONH-arylalkyl), carbonyl, carboxyl, carboxylic acid, carboxylic acid anhydride, carboxylic acid chloride, cyano, ester, epoxide, ether (e.g., methoxy, ethoxy), guanidino, halo, haloalkyl (for example —CCl₃, —CF₃, —C(CF₃)₃), heteroalkyl, hemiacetal, imine (primary and secondary), isocyanate, isothiocyanate, ketone, nitrile, nitro, oxo, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) and urea (—NHCONH-alkyl-).

Some compounds of the present invention can exist in a tautomeric form which is also intended to be encompassed within the scope of the present invention. “Tautomers” refers to compounds whose structures differ markedly in the arrangement of atoms, but which exist in easy and rapid equilibrium. It is to be understood that compounds of present invention may be depicted as different tautomers. It should also be understood that when compounds have tautomeric forms, all tautomeric forms are intended to be within the scope of the present invention, and the naming of the compounds does not exclude any tautomeric form. Tautomers exist as mixtures of a tautomeric set in solution. In solid form, usually one tautomer predominates. Even though one tautomer may be described, the present invention includes all tautomers of the compounds disclosed herein. A tautomer is one of two or more structural isomers that exist in equilibrium and are readily converted from one isomeric form to another. This reaction results in the formal migration of a hydrogen atom accompanied by a shift of adjacent conjugated double bonds. In solutions where tautomerization is possible, a chemical equilibrium of the tautomers can be reached. The exact ratio of the tautomers depends on several factors, including temperature, solvent, and pH. The concept of tautomers that are interconvertible by tautomerizations is called tautomerism. Tautomerizations are catalyzed by: Base: 1. deprotonation; 2. formation of a delocalized anion (e.g., an enolate); 3. protonation at a different position of the anion; Acid: 1. protonation; 2. formation of a delocalized cation; 3. deprotonation at a different position adjacent to the cation.

As used herein the terms “subject” and “patient” are well-recognized in the art, and, are used herein to refer to a mammal, and most preferably a human. In some embodiments, the subject is a subject in need of treatment or a subject being infected by a coronavirus, who is likely to benefit from a treatment with combination therapy of the present invention. The term does not denote a particular age or sex. Thus, adult and newborn subjects, whether male or female, are intended to be covered.

As used herein the term “pharmaceutically acceptable excipients and/or carriers” means that the compositions or components thereof so described are suitable for use in contact with a mammal body, preferably human body, or suitable for any other means of administration to human body without undue toxicity, incompatibility, instability, irritability, allergic response, and the like.

The term includes any and all solvents, dispersion media, coatings, antibacterial and antifungal agents, isotonic and absorption delaying agents and the like. The use of such media and agents for pharmaceutically active substances is well known in the art. In addition, various adjuvants such as are commonly used in the art may be included. Considerations for the inclusion of various components in pharmaceutical compositions are described, for example, in Gilman et al. (Eds.) (1990); Goodman and Gilman's: The Pharmacological Basis of Therapeutics, 8th Ed., Pergamon Press, which is incorporated herein by reference in its entirety.

The term “treat” and its grammatical variants (for example “to treat,” “treating,” and “treatment”) refer to administration of the combination therapy of the invention to a subject with the purpose of ameliorating and/or reducing the lung inflammation, resulting from MERS-CoV and/or SARS-CoV virus infection, during and following the viral infection of a subject. Such amelioration may be partial or complete. In the present context, treatment entails administering the pharmaceutical combination of the invention to a subject or the co-administration of a flavonoid compound and a sphingosine-1-phosphate lyase inhibitor (S1PLI) to a subject.

The term “therapeutically effective amount,” as used herein, refers to any amount of a specific component or combination of components that will cause a reduction of symptoms, disappearance of the symptoms or relief from symptoms related to for example lung inflammation resulting from MERS-CoV and/or SARS-CoV virus infection (coronavirus infection), when applied, either once, or repeatedly over time. Therapeutically effective amounts can be readily determined by persons skilled in the art using routine experimentation and using tests and measures commonly employed in the art, or can be based upon the subjective response of patients undergoing treatment.

In the context of the present invention, SARS-CoV virus includes SARS-CoV-1 virus and SARS-CoV-2 virus.

An aspect of the invention provides a pharmaceutical combination of a flavonoid compound and a sphingosine-1-phosphate lyase inhibitor (S1PLI) for simultaneous, separate or sequential administration, wherein the flavonoid compound is selected from the group comprising dihydroquercetin (DHQ), quercetin, astilbin, dihydrokaempferol, butin, eriodictyol, hesperetin, hesperidin, homoeriodictyol, isosakuranetin, naringenin, naringin, pinocembrin, poncirin, sakuranetin, sakuranin, sterubin, epigallocatechin gallate, catechin, epicatechin; preferably the flavonoid compound is selected from the group comprising dihydroquercetin (DHQ), quercetin, astilbin, epigallocatechin gallate, catechin and epicatechin; more preferably the flavonoid compound is selected from the group comprising dihydroquercetin (DHQ), quercetin, astilbin and epigallocatechin gallate;
wherein the sphingosine-1-phosphate lyase inhibitor (S1PLI) is a compound of Formula

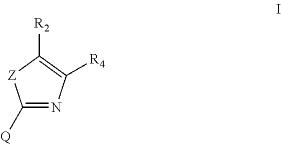

wherein
Z is selected from the group comprising O, S, NH, preferably Z is O or S or preferably Z is NH;
Q is

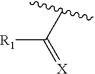

or optionally substituted heterocycle; preferably Q is

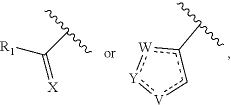

more preferably Q is

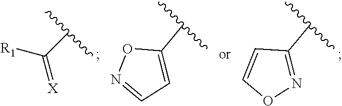

X is O or NR₃; preferably X is NR₃;
each of W, Y, V is independently selected from the group comprising CH₂, CH, N, NH, O or S;
R₁ is selected from the group comprising OR_{A}, NHOH, hydrogen, optionally substituted alkyl, optionally substituted aryl, optionally substituted alkylaryl, optionally substituted arylalkyl, optionally substituted heteroalkyl, optionally substituted heterocycle, optionally substituted alkylheterocycle, optionally substituted heterocyclealkyl; preferably R₁ is C₁-C₅ alkyl; more preferably R₁ is —CH₃;
R₂ is selected from the group comprising OR_{B}, C(O)OR_{B}, hydrogen, halogen, nitrile, optionally substituted hydroxyalkyl, optionally substituted alkyl, optionally substituted aryl, optionally substituted alkylaryl, optionally substituted arylalkyl, optionally substituted heteroalkyl, optionally substituted heterocycle, optionally substituted alkylheterocycle, optionally substituted heterocyclealkyl; preferably, R₂ is hydrogen or —(CH₂)ₙ—OH, wherein n is 1 to 5, preferably n is 1;
R₃ is selected from the group comprising OR_{C}, N(R_{C})₂, NHC(O)R_{C}, NHSO₂R_{C}, hydrogen; preferably, R₃ is —OH;
R₄ is selected from the group comprising OR_{D}, OC(O)R_{D}, N(R_{E})₂, hydrogen, halogen, optionally substituted hydroxyalkyl, optionally substituted alkyl, optionally substituted aryl, optionally substituted alkylaryl, optionally substituted arylalkyl, optionally substituted heteroalkyl, optionally substituted heterocycle, optionally substituted alkylheterocycle, optionally substituted heterocyclealkyl; preferably R₄ is hydrogen, —(CH₂)ₙ—OH, wherein n is 1 to 5, preferably n is 1 or C₄ hydroxyalkyl (such as tetrahydroxybutyl);
each of R_{A}, R_{B}, R_{C}, R_{D}, and R_{E} is independently selected from the group comprising hydrogen, optionally substituted alkyl, optionally substituted aryl, optionally substituted alkylaryl, optionally substituted arylalkyl, optionally substituted heteroalkyl, optionally substituted heterocycle, optionally substituted alkylheterocycle, or optionally substituted heterocyclealkyl.

In some embodiments of the sphingosine-1-phosphate lyase inhibitor (S1PLI) compound of Formula I, R₁ is selected from the group comprising OR_{A}, NHOH, hydrogen, alkyl, aryl, alkylaryl, arylalkyl, heteroalkyl, heterocycle, alkylheterocycle, heterocyclealkyl; preferably R₁ is C₁-C₅ alkyl; more preferably R₁ is —CH₃.

In some embodiments of the sphingosine-1-phosphate lyase inhibitor (S1PLI) compound of Formula I, R₂ is selected from the group comprising OR_{B}, C(O)OR_{B}, hydrogen, halogen, nitrile, hydroxyalkyl, alkyl, aryl, alkylaryl, arylalkyl, heteroalkyl, heterocycle, alkylheterocycle, heterocyclealkyl; preferably, R₂ is hydrogen or —(CH₂)ₙ—OH, wherein n is 1 to 5, preferably n is 1.

In some embodiments of the sphingosine-1-phosphate lyase inhibitor (S1PLI) compound of Formula I, R₄ is selected from the group comprising OR_{D}, OC(O)R_{D}, N(R_{E})₂, hydrogen, halogen, hydroxyalkyl, alkyl, aryl, alkylaryl, arylalkyl, heteroalkyl, heterocycle, alkylheterocycle, heterocyclealkyl; preferably R₄ is hydrogen, —(CH₂)ₙ—OH, wherein n is 1 to 5, preferably n is 1 or C₄ hydroxyalkyl (such as tetrahydroxybutyl).

In some embodiments of the sphingosine-1-phosphate lyase inhibitor (S1PLI) compound of Formula I, each of R_{A}, R_{B}, R_{C}, R_{D}, and R_{E} is independently selected from the group comprising hydrogen, alkyl, aryl, alkylaryl, arylalkyl, heteroalkyl, heterocycle, alkylheterocycle, or heterocyclealkyl.

In the context of the present invention, it is important that the flavonoid compound has the C ring saturated, i.e. no double bond between positions 2 and 3 (see below the compound of formula II). Thus these chemical compounds do not interact with singlet oxygen to generate a toxic reactive endoperoxide. The flavonoid compounds can be multi-hydroxylated, and several hydroxyl groups can be glycosylated and/or methylated. Catechins and derivatives thereof, such as epicatechin, have two benzene rings (called the A- and B-rings) and a dihydropyran heterocycle (the C-ring) with a hydroxyl group on carbon 3. The A ring is similar to a resorcinol moiety while the B ring is similar to a catechol moiety. There are two chiral centres on the molecule on carbons 2 and 3 (see below the compound of formula II). Therefore, it has four diastereoisomers. Two of the isomers are in trans configuration and are called catechin and the other two are in cis configuration and are called epicatechin. Preferably catechin is (+)-catechin and a derivative thereof. Derivatives of (+)-catechin are for example (+)-catechin C, (+)-gallocatechine GC.

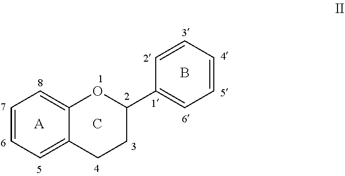

The invention further provides a kit comprising combination of the flavonoid compound of the invention, the sphingosine-1-phosphate lyase inhibitor (S1PLI) compound of the invention and an information leaflet containing written instructions for administering the flavonoid compound and S1PLI compound.

It will be appreciated that the individual compounds of the pharmaceutical combination of the invention may be administered simultaneously, either in the same formulation (composition) or different pharmaceutical formulations (compositions), separately or sequentially. If there is separate or sequential administration, the delay in administering the individual compounds should not be such as to lose the benefit of any synergistic therapeutic effect of the combination of the flavonoid compound of the invention and the sphingosine-1-phosphate lyase inhibitor (S1PLI) compound of the invention.

In some embodiments, the sphingosine-1-phosphate lyase inhibitor (S1PLI) compound of the invention of Formula I is selected from the group comprising

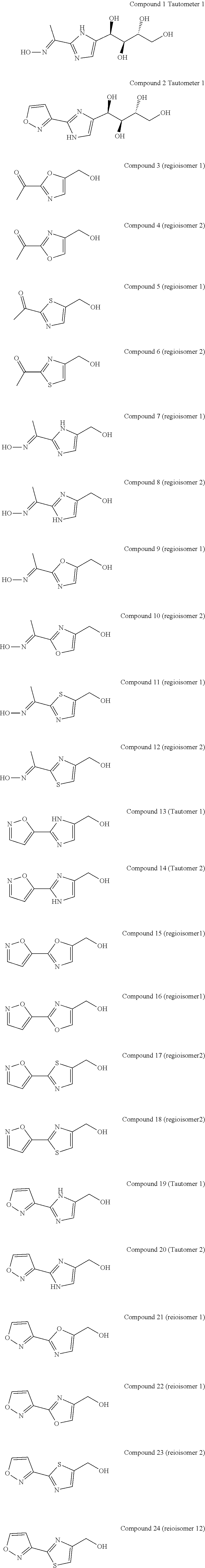

In some embodiments, the sphingosine-1-phosphate lyase inhibitor (S1PLI) compound of the invention of Formula I is

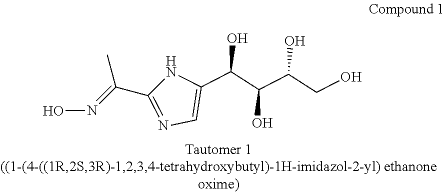 [SMILES: C/C(=N\O)c1ncc([C@@H](O)[C@H](O)[C@H](O)CO)[nH]1]

The sphingosine-1-phosphate lyase inhibitor (S1PLI) compounds of the invention can be prepared by methods known in the art.

The compounds of formula I or tautomers thereof, or pharmaceutically acceptable salts of said compounds or tautomers disclosed herein can have asymmetric centres. The compounds of Formula I or tautomers thereof, or pharmaceutically acceptable salts of said compounds or tautomers of the present invention containing an asymmetrically substituted atom can be isolated in optically active or racemic forms. It is well known in the art how to prepare optically active forms, such as by resolution of racemic forms or by synthesis from optically active starting materials. Cis and trans geometric isomers of the compounds of Formula I or tautomers thereof, or pharmaceutically acceptable salts of said compounds or tautomers of the present invention are described and can be isolated as a mixture of isomers or as separate isomeric forms. All chiral, diastereomeric, racemic, and geometric isomeric forms of a structure are intended, unless specific stereochemistry or isomeric form is specifically indicated. All processes used to prepare compounds of Formula I or tautomers thereof, or pharmaceutically acceptable salts of said compounds or tautomers of the present invention and intermediates made herein are considered to be part of the present invention. All tautomers of shown or described compounds are also considered to be part of the present invention.

Specifically, the compounds of Formula I can contain one or more asymmetric centres and can thus occur as racemates and racemic mixtures, single enantiomers, diastereomeric mixtures and individual diastereomers. The present invention is meant to comprehend all such isomeric forms of the compounds of Formula I. The compounds of Formula I may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration. Alternatively, any stereoisomer of a compound of the general structural Formula I may be obtained by stereospecific synthesis using optically pure starting materials or reagents of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base.

The diasteromeric derivatives (see Table 1) may then be converted to the pure enantiomers by cleavage of the added chiral residue. The racemic mixture of the compounds can also be separated directly by chromatographic methods utilizing chiral stationary phases, which methods are well known in the art.

TABLE 1
Tautomers and regioisomers of the compounds of the invention

| Compound No. | Tautomer (Tau.) | Regioisomer (Reg.) | Ring |
|---|---|---|---|
| 3 - ACB1903 | | 1 | oxazole |
| 4 - ACB1904 | | 2 | oxazole |
| 5 - ACB1905 | | 1 | thiazole |
| 6 - ACB1906 | | 2 | thiazole |
| 7 - ACB1907 | 1 | | imidazole |
| 8 - ACB1908 | 2 | | imidazole |
| 9 - ACB1909 | | 1 | oxazole |
| 10 - ACB1910 | | 2 | oxazole |
| 11 - ACB1911 | | 1 | thiazole |
| 12 - ACB1912 | | 2 | thiazole |
| 13 - ACB1913 | 1 | | imidazole |
| 14 - ACB1914 | 2 | | imidazole |
| 15 - ACB1915 | | 1 | oxazole |
| 16 - ACB1916 | | 2 | oxazole |
| 17 - ACB1917 | | 1 | thiazole |
| 18 - ACB1918 | | 2 | thiazole |
| 19 - ACB1919 | 1 | | imidazole |
| 20 - ACB1920 | 2 | | imidazole |
| 21 - ACB1921 | | 1 | oxazole |
| 22 - ACB1922 | | 2 | oxazole |
| 23 - ACB1923 | | 1 | thiazole |
| 24 - ACB1924 | | 2 | thiazole |

The compounds of formula (I) or tautomers thereof, or pharmaceutically acceptable salts of said compounds or tautomers disclosed herein have at least one side chain that is typically an aliphatic primary alcohol such as —(CH₂)ₙ—OH, were n is preferably 1, that can be phosphorylated by PKA and/or by other enzymes, such as pyridoxal kinase, during metabolism of the compounds of the invention in the human body.

The sphingosine-1-phosphate lyase inhibitor (S1PLI) are small chemical molecules which inhibits the catalytic activity of S1P lyase, the major enzyme involved in the terminal degradation of S1P into 2-hexadecanal and phosphoethanolamine. S1P and its metabolites are known modulators of many aspects of the immune responses, including phagocytosis, inflammation, pathogen persistence, cell death and chemotaxis acting either in an extracellular or intracellular manner. A protective role for the S1P in lung pathologies such as LPS-induced lung injury (sepsis), pulmonary fibrosis and bronchopulmonary dysplasias has been well described in the literature [Ebenezer et al, 2017, Zhao et al, 2011, Huang et al, 2015). Animal models indicate that administration of S1P, its analogues or the administration of S1PL inhibitors reduced vascular leakage and pulmonary edema in sepsis-induced lung injury, while reducing pro-inflammatory mediators (Ebenezer et al, 2017). Similarly, inhibition of S1PL activity during experimental ventilator induced lung injury reduced levels of neutrophils and macrophages into the lung (Suryadevara et al, 2018).

Dihydroquercetin (DHQ) 3,3′,4′,5,7-Pentahydroxyflavone dihydrate, 2-(3,4-Dihydroxyphenyl)-3,5,7-trihydroxy-4H-1-benzopyran-4-one dehydrate (Formula: C₁₅H₁₀O₇.2H₂O, molecular weight 338.27), referred to as Taxifolin, is a natural compound of the flavonoid family characterized by a great chemical stability with conserved significant biological and pharmacological properties. From an historical point of view, DHQ was first identified as a powerful antioxidant in the 1940s. In 1958 the Journal of the American Pharmaceutical Association published research establishing its safety and by the mid-1960s DHQ was being widely investigated for use as a natural preservative in all kinds of foods. A Russian company has applied to the Food Standards Agency for approval to market DHQ as a novel food ingredient which follow the Sanitary Rules and Norm's 2.3.2.1078-01. DHQ is extracted from a type of larch wood and has been marketed in Russia and the US for 15-20 years as a food supplement. The company, Ametis JSG, is seeking an authorization to market DHQ as a dietary supplement in dairy, meat and confectionery products, as well as in oil and fats, and alcoholic and non-alcoholic beverages. In USA DHQ was tested by Biotec Center, Foran Hall, Cook College, 59 Dudley Road, New Brunswick, N.J. 08901-8520 USA. The product is included in list of FDA.

Astilbin is readily transformed to DHQ following the ingestion.

DHQ/astilbin displays anticancer, anti-oxidative, anti-inflammatory, and immunosuppressive activity. In vitro, DHQ inhibits Th17 cell differentiation and IL-17 secretion of isolated T cells, and inhibits Jak/Stat3 signaling in Th17 cells, while up-regulating Stat3 inhibitor SCOSE3 expression. DHQ has been reported to possess multiple clinically relevant bioactivities, including antioxidant, anti-inflammatory, anti-arthritic, and anti-diabetic nephropathy properties. DHQ/astilbin is reported to reduce activation of both T and B cells in lupus-prone mice. It significantly inhibits inflammatory responses and keratinocyte over-proliferation in a mouse model of imiquimod (IMQ)-induced psoriasis. DHQ is a potent inhibitor of NADPH oxidase, resulting in the inhibition of neutrophils oxidative burst.

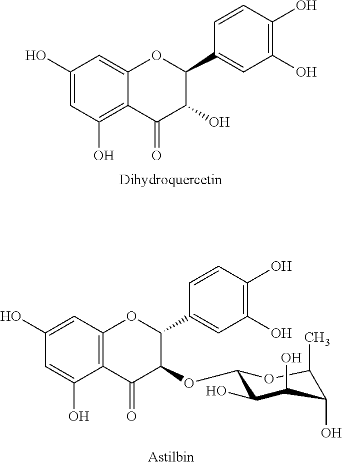

Another aspect of the present invention provides a pharmaceutical composition comprising the flavonoid compound of the invention and pharmaceutically acceptable excipients and/or carriers and another pharmaceutical composition comprising the sphingosine-1-phosphate lyase inhibitor (S1PLI) compound of the invention and pharmaceutically acceptable excipients and/or carriers.

Certain pharmaceutical compositions of the invention are single unit dosage forms suitable for oral or mucosal (such as nasal, sublingual, vaginal, buccal, or rectal) administration to a patient. Examples of dosage forms include, but are not limited to tablets, caplets, capsules, such as soft elastic gelatine capsules, cachets, troches, lozenges, dispersions, suppositories, powders, solutions (fluid solutions), aerosols (such as nasal sprays or inhalers), liquid dosage forms suitable for oral or mucosal administration to a patient, including suspensions (such as aqueous or non-aqueous liquid suspensions, oil-in-water emulsions, or a water-in-oil liquid emulsions), solutions, and elixirs.

The formulation of the pharmaceutical composition of the invention should suit the mode of administration. For example, oral administration requires enteric coatings to protect the compounds of this invention from degradation within the gastrointestinal tract. Similarly, a formulation may contain ingredients that facilitate delivery of the active ingredient(s) to the site of action. For example, compounds may be administered in liposomal formulations, in order to protect them from degradative enzymes, facilitate transport in circulatory system, and effect delivery across cell membranes to intracellular sites.

The pharmaceutical compositions of the invention suitable for oral administration can be presented as discrete dosage forms, such as, but are not limited to, tablets (such as chewable tablets), caplets, capsules, and liquids (such as flavoured syrups). Such dosage forms contain predetermined amounts of active ingredients, and may be prepared by methods of pharmacy well known to those skilled in the art. See, e.g., Remington's Pharmaceutical Sciences, 18th ed., Mack Publishing, Easton Pa. (1990).

Typical oral dosage forms are prepared by combining the compounds of the invention in an intimate admixture with at least one excipient according to conventional pharmaceutical compounding techniques. Excipients can take a wide variety of forms depending on the form of preparation desired for administration.

Because of their ease of administration, tablets and capsules represent an advantageous oral dosage unit form. If desired, tablets can be coated by standard aqueous or nonaqueous techniques. Such dosage forms can be prepared by conventional methods of pharmacy. In general, pharmaceutical compositions and dosage forms are prepared by uniformly and intimately admixing the compound of the invention with liquid carriers, finely divided solid carriers, or both, and then shaping the product into the desired presentation if necessary. Disintegrants may be incorporated in solid dosage forms to facility rapid dissolution. Lubricants may also be incorporated to facilitate the manufacture of dosage forms (such as tablets). For example pharmaceutically acceptable excipients particularly suitable for use in conjunction with tablets include, for example, inert diluents, such as celluloses, calcium or sodium carbonate, lactose, calcium or sodium phosphate; disintegrating agents, such as cross-linked povidone, maize starch, or alginic acid; binding agents, such as povidone, starch, gelatin or acacia; and lubricating agents, such as magnesium stearate, stearic acid or talc.

Other pharmaceutical compositions of the invention are parenteral dosage forms administered to patients by various routes including, but not limited to, subcutaneous, intravenous (including bolus injection), intramuscular, and intraarterial. Because their administration typically bypasses patients' natural defenses against contaminants, parenteral dosage forms are specifically sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to a person skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

The composition, shape, and type of a dosage form of the pharmaceutical composition of the invention will vary depending on its use. For example, a dosage form used in the acute treatment of a disease, such as infectious diseases, may contain larger amounts of one or more of the compounds of the invention than a dosage form used in the chronic treatment of the same disease.

In one embodiment of the invention, the pharmaceutical composition of the invention is suitable for per os administration. In an embodiment, the pharmaceutical composition of the invention is a capsule comprising from 10 to 200 mg DHQ or astilbin, preferably 100 mg DHQ or astilbin, and vitamin C at the same dosage. In another embodiment, the pharmaceutical composition of the invention is a capsule comprising from 10 to 200 mg S1PLI, preferably 100 mg S1PLI.

According to one embodiment, patients will ingest one capsule containing DHQ plus one capsule containing S1PLI either once or twice a day depending on the clinical evolution of the disease (infection).

In some embodiment of the invention, the pharmaceutical composition of the invention is suitable for intra gastric administration. In an embodiment, the pharmaceutical composition of the invention is a fluid solution; one fluid solution containing DHQ or astilbin and vitamin C and another fluid solution containing S1PLI.

The present invention relates to a method of treatment relating to ameliorating and/or reducing the lung inflammation, resulting from MERS-CoV and/or SARS-CoV, during and following the viral infection of a subject. MERS-CoV (Middle Eastern respiratory syndrome coronavirus) and SARS-CoV (Severe acute respiratory syndrome coronavirus), including SARS-CoV-1 and SARS-CoV-2, can cause severe acute respiratory syndrome, whereby the physiological damage and risk of mortality is due to exacerbated and uncontrolled inflammation, such as lung inflammation, rather than the viral load. One of the objectives of the method of ameliorating and/or reducing the lung inflammation according to the invention is to decrease the activation of neutrophils, and other myeloid cells, and impact cellular recruitment to the site of infection.

Another objective of the method of ameliorating and/or reducing the lung inflammation according to the invention is to treat long term respiratory complications and/or avoid lasting lung damages, typically the lung damages that follow the virus infection of a subject. The method of ameliorating and/or reducing the lung inflammation according to the invention consists in a combination of a flavonoid compound of the invention, such as dihydroquercetin (DHQ), quercetin, astilbin and epigallocatechin gallate, and a sphingosine-1-phosphate lyase inhibitor (S1PL inhibitor or S1PLI) compound of the invention. Indeed, DHQ is a potent inhibitor a neutrophil and myeloid cell activation, while S1PL inhibitor induces a sequestration of T lymphocytes in lymph nodes and impacts the ability to generate pathological pro-inflammatory responses. Taken together, these pharmacological effects result in the decrease of lung inflammation (and respiratory distress). This treatment, i.e. this method of ameliorating and/or reducing the lung inflammation, should be taken during the acute virus infection of a subject (i.e. during the virus infection of a subject) and continued after the decrease of the viremia in a subject (i.e. following the virus infection of a subject).

An aspect of the invention relates to a method for ameliorating and/or reducing the lung inflammation, resulting from MERS-CoV and/or SARS-CoV virus infection, during and following the virus infection of a subject, comprises administering to the subject in need thereof the pharmaceutical combination of the invention.

An aspect of the invention relates to a pharmaceutical combination of the invention for use in a method for ameliorating and/or reducing the lung inflammation, resulting from MERS-CoV and/or SARS-CoV virus infection, during and following the virus infection of a subject.

Another aspect of the invention relates to a method for ameliorating and/or reducing the lung inflammation, resulting from MERS-CoV and/or SARS-CoV virus infection, during and following the virus infection of a subject, the method comprising co-administering to the subject in need thereof a therapeutically effective amounts of a flavonoid compound and a sphingosine-1-phosphate lyase inhibitor (S1PL inhibitor).

Another aspect of the invention provides a sphingosine-1-phosphate lyase inhibitor (S1PL inhibitor) for use in a method for ameliorating and/or reducing the lung inflammation, resulting from MERS-CoV and/or SARS-CoV virus infection, during and following the virus infection of a subject, the method comprising co-administering to the subject in need thereof a therapeutically effective amounts of a flavonoid compound and a sphingosine-1-phosphate lyase inhibitor (S1PL inhibitor).

In one embodiment of the method for ameliorating and/or reducing the lung inflammation of the invention, the flavonoid compound is selected from the group comprising dihydroquercetin (DHQ), quercetin, astilbin, dihydrokaempferol, butin, eriodictyol, hesperetin, hesperidin, homoeriodictyol, isosakuranetin, naringenin, naringin, pinocembrin, poncirin, sakuranetin, sakuranin, sterubin, epigallocatechin gallate, catechin, epicatechin; preferably the flavonoid compound is selected from the group comprising dihydroquercetin (DHQ), quercetin, astilbin, epigallocatechin gallate, catechin and epicatechin; more preferably the flavonoid compound is selected from the group comprising dihydroquercetin (DHQ), quercetin, astilbin and epigallocatechin gallate.

In other embodiment of the method for ameliorating and/or reducing the lung inflammation of the invention, the sphingosine-1-phosphate lyase inhibitor (S1PL inhibitor) is a compound of Formula I

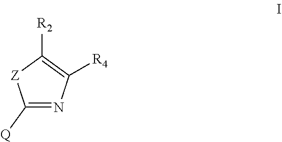

wherein
Z is selected from the group comprising O, S, NH, preferably Z is O or S or preferably Z is NH;
Q is

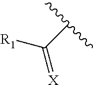

or optionally substituted heterocycle; preferably Q is

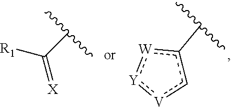

more preferably Q is

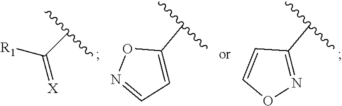

X is O or NR₃; preferably X is NR₃;
each of W, Y, V is independently selected from the group comprising CH₂, CH, N, NH, O or S;
R₁ is selected from the group comprising OR_{A}, NHOH, hydrogen, optionally substituted alkyl, optionally substituted aryl, optionally substituted alkyl aryl, optionally substituted arylalkyl, optionally substituted hetero alkyl, optionally substituted heterocycle, optionally substituted alkyl heterocycle, optionally substituted heterocycle alkyl; preferably R₁ is C₁-C₅ alkyl or hydrogen; more preferably R₁ is —CH₃ or hydrogen;
R₂ is selected from the group comprising OR_{B}, C(O)OR_{B}, hydrogen, halogen, nitrile, optionally substituted hydroxyalkyl, optionally substituted alkyl, optionally substituted aryl, optionally substituted alkylaryl, optionally substituted arylalkyl, optionally substituted hetero alkyl, optionally substituted heterocycle, optionally substituted alkyl heterocycle, optionally substituted heterocycle alkyl; preferably, R₂ is hydrogen or —(CH₂)ₙ—OH, wherein n is 1 to 5, preferably n is 1;
R₃ is selected from the group comprising OR_{C}, N(R_{C})₂, NHC(O)R_{C}, NHSO₂R_{C}, hydrogen; preferably, R₃ is —OH;
R₄ is selected from the group comprising OR_{D}, OC(O)R_{D}, N(R_{E})₂, hydrogen, halogen, optionally substituted hydroxyalkyl, optionally substituted alkyl, optionally substituted aryl, optionally substituted alkylaryl, optionally substituted arylalkyl, optionally substituted heteroalkyl, optionally substituted heterocycle, optionally substituted alkylheterocycle, optionally substituted heterocyclealkyl; preferably R₄ is hydrogen, —(CH₂)ₙ—OH, wherein n is 1 to 5, preferably n is 1, or C₄ hydroxyalkyl (such as tetrahydroxybutyl);
each of R_{A}, R_{B}, R_{C}, R_{D}, and R_{E} is independently selected from the group comprising hydrogen, optionally substituted alkyl, optionally substituted aryl, optionally substituted alkylaryl, optionally substituted arylalkyl, optionally substituted heteroalkyl, optionally substituted heterocycle, optionally substituted alkylheterocycle, or optionally substituted heterocyclealkyl.

As described above, the method for ameliorating and/or reducing the lung inflammation, resulting from MERS-CoV and/or SARS-CoV virus infection, during and following the virus infection include co-administering the flavonoid compound of the invention and the sphingosine-1-phosphate lyase inhibitor (S1PLI) compound of the invention. By “co-administration” or “co-administering”, it is meant that the flavonoid compound of the invention and the sphingosine-1-phosphate lyase inhibitor (S1PLI) compound of the invention are administered in such a manner that administration of the flavonoid compound of the invention has an effect on the efficacy of the treatment of the sphingosine-1-phosphate lyase inhibitor (S1PLI) compound of the invention, such as to provide a synergistic therapeutic effect. Thus in one embodiment, the flavonoid compound of the invention and the sphingosine-1-phosphate lyase inhibitor (S1PLI) compound of the invention are administered simultaneously. In one such embodiment, administration in combination is accomplished by combining the flavonoid compound of the invention and the sphingosine-1-phosphate lyase inhibitor (S1PLI) compound of the invention in a single dosage form, unit and/or kit. In another embodiment, the flavonoid compound of the invention and the sphingosine-1-phosphate lyase inhibitor (S1PLI) compound of the invention are administered sequentially. In one embodiment the flavonoid compound of the invention and the sphingosine-1-phosphate lyase inhibitor (S1PLI) compound of the invention are administered through the same route, such as orally. In another embodiment, the flavonoid compound of the invention and the sphingosine-1-phosphate lyase inhibitor (S1PLI) compound of the invention are administered through different routes, such as one being administered orally and another being administered parenterally. In some embodiments, the time period between administration of the flavonoid compound of the invention and administration of the co-administered sphingosine-1-phosphate lyase inhibitor (S1PLI) compound of the invention can be about 1 hour, 2 hours, 3 hours, 5 hours, 8 hours, 10 hours, 12 hours, 15 hours, 18 hours, 20 hours, 24 hours, 36 hours, 48 hours, 3 days, 4 days, 5 days, 6 days, 7 days, 10 days, 14 days, 21 days, 28 days, or 30 days.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended claims, and all changes which come within the meaning and range of equivalency are intended to be embraced therein.

The foregoing description will be more fully understood with reference to the following Examples. Such Examples, are, however, exemplary of methods of practising the present invention and are not intended to limit the application and the scope of the invention.

### Examples

Flavonoid Dihydroquercetin (DHQ) and Related Compounds

Flavonoids are the largest group of naturally occurring polyphenolic compounds which have shown diverse biological activities depending of their chemical structure including anti-viral activities against a variety of viruses and potent immune-modulating and inflammatory activities. Among flavonoids, Quercetin, and its bioavailable derivatives such as isoquercetin, offer good perspectives to develop new therapeutics to treat cancer, viral infection and pathogenic inflammation (reviewed in Li et al, 2016). Flavonoids have potent anti-inflammatory activities. Recently, Zaragoza et al. have reported that Quercetin decreases the production of the major inflammatory cytokines: TNFα, IL-6, IL-8 and IL-10 in LPS-stimulated whole blood (Zaragoza et al. 2020). This result strongly supports that flavonoid has a potential therapeutic effect in the inflammatory process. The inventor of the present invention found that DHQ and quercetin display a strong inhibitory effect on the activation of neutrophils stimulated by LPS (lipopolysaccharide) or PMA (phorbolmyristate acetate).

Effect of DHQ on Neutrophils Activation

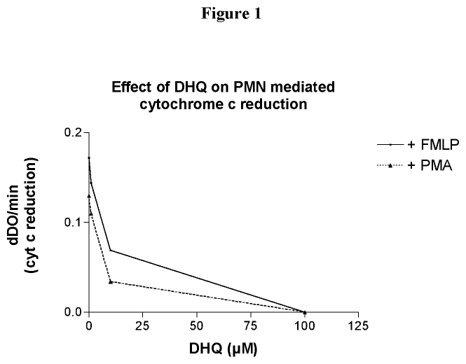

When activated by opsonized bacteria, zymozan, FMLP (Formyl-Methionyl-Leucyl-Phenylalanine) or PMA (phorbolmyristate acetate), neutrophils undergo an oxidative burst resulting in the production of superoxide anion and subsequent hydrogen peroxide. The oxidative burst is mainly mediated by the NADPH oxidase activation, enzyme responsible of the superoxide anion production. Superoxide anion can be quantified by the measurement of the cytochrome c reduction as shown in Table 2 and FIG. 1. Data indicated in Table 2 and FIG. 1 show that the addition of DHQ strongly inhibits cytochrome c reduction. This effect may come from either a direct anion superoxide scavenging or from the inhibition of NADPH oxidase activation.

TABLE 2
Inhibitory effect of DHQ on the cytochrome c reduction
(anion superoxide production) resulting from the neutrophils
activation by PMA or FLMP. It should be noticed that
DHQ induces a significant cytochrome c reduction.

| Neutrophils | | DHQ: | DHQ: | DHQ: |
|---|---|---|---|---|
| status: | Control | 1 μM | 10 μM | 100 μM |
| Resting (dDO/min) | 0.004 | 0.000 | 0.036 | 0.086 |
| +FMLP (dDO/min) | 0.172 | 0.144 | 0.105 | 0.040 |
| +PMA (dDO/min) | 0.130 | 0.110 | 0.070 | 0.021 |

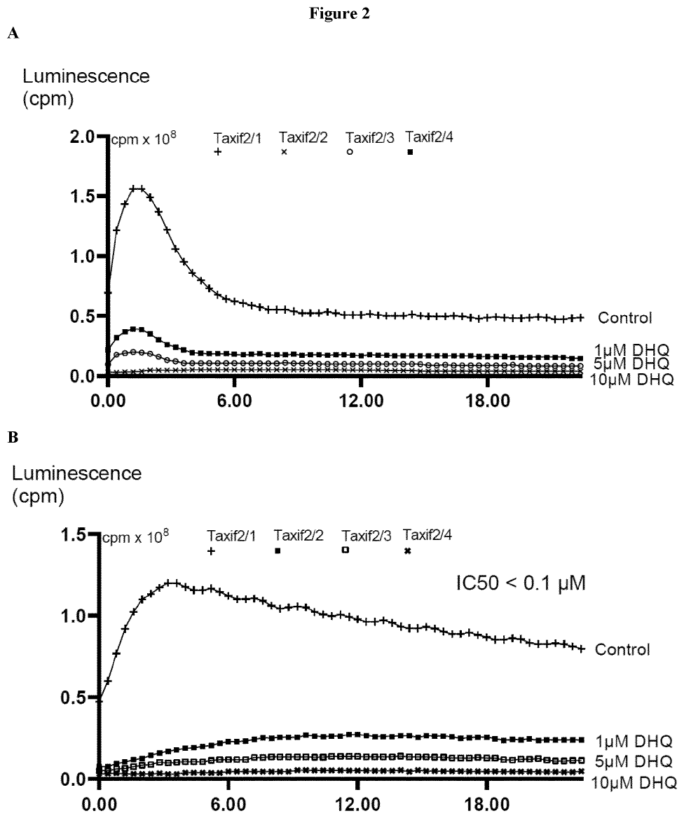

In order to further investigate the effect of DHQ on the oxidative burst of neutrophils, the measurement of the ROS production by chemoluminescence has been used which detect both surperoxide anion and hydrogen peroxide. The results of FIG. 2 show that DHQ strongly inhibits the ROS production by neutrophils activated either by PMA or FMLP. This inhibitory effect can be considered as very efficient regarding the fact that the addition of 1p M in the assay medium results in around 80% inhibition of the ROS production in both experiments. The inhibition is not due to a ROS scavenging (as measured by cyt. C reduction) but due to the inhibition of NADPH oxidase.

TABLE 3
Experimental data corresponding to the graph drawn in the FIG. 2. Neutrophils were
triggered by PMA(100 ng/ml) in the presence of different concentrations of DHQ (0,
1, 10 and 100 μM) at 37° C. in Hanks buffer containing 10 μM luminol and
chemiluminescence was measured by a chemiluminometer. Total chemiluminescence counts
during 22.21 min. (integrals) corresponding to total ROS production were determined.
Report
Taxif2
8 Samples
Measuring Time: 22.21 min
Integration Time: 0.00 to 22.21 min

| Sample | Peak max | Slope max | T. Slope | T. half | T. max | T. half | Smoot |
|---|---|---|---|---|---|---|---|
| Integral | cpm | cpm | max | (rise) | (peak) | (fall) | Facto |
| 2.150E+09 | 1.196E+08 | 6.991E+07 | 0.67 | 0.22 | 3.33 | > | 0 |
| 3.573E+07 | 2.260E+06 | 1.033E+06 | 4.66 | 4.89 | 11.55 | > | 0 |
| 2.072E+08 | 1.134E+07 | 4.380E+06 | 1.55 | 2.22 | 9.55 | > | 0 |
| 4.509E+08 | 2.430E+07 | 9.790E+06 | 1.78 | 2.44 | 11.77 | > | 0 |

TABLE 4
Experimental data corresponding to the graph drawn in the FIG. 2B. Neutrophils were
triggered by FMLP(10−6 Ml) in the presence of different concentrations of DHQ
(0, 1, 10 and 100 μM) at 37° C. in Hanks buffer containing 10 μM luminol
and chemiluminescence was measured by a chemiluminometer. Total chemiluminescence
counts during 22.21 min. (integrals) corresponding to total ROS production were determined.

| | | cpm | cpm | max | (rise) | (peak) | (fall) | Factor |
|---|---|---|---|---|---|---|---|---|
| 1 | 1.453E+09 | 1.571E+08 | 9.691E+07 | 0.22 | < | 1.55 | 4.44 | 0 |
| 2 | 4.329E+07 | 2.533E+06 | 1.464E+06 | 2.00 | 1.55 | 6.00 | > | 0 |
| 3 | 1.867E+08 | 1.770E+07 | 1.206E+07 | 0.22 | < | 1.33 | 3.55 | 0 |
| 4 | 3.838E+08 | 3.731E+07 | 2.492E+07 | 0.22 | < | 1.33 | 3.78 | 0 |

Report
Taxif2
8 Samples
Measuring Time: 22.21 min
Integration Time: 0.00 to 22.21 min

| | | Peak max | Slope max | T. Slope | T. half | T. max | T. half | Smoot |
|---|---|---|---|---|---|---|---|---|
| Sample | Integral | cpm | cpm | max | (rise) | (peak) | (fall) | Facto |
| 1 | 1.453E+09 | 1.571E+08 | 9.691E+07 | 0.22 | < | 1.55 | 4.44 | 0 |
| 2 | 4.329E+07 | 2.533E+06 | 1.464E+06 | 2.00 | 1.55 | 6.00 | > | 0 |
| 3 | 1.867E+08 | 1.770E+07 | 1.206E+07 | 0.22 | < | 1.33 | 3.55 | 0 |
| 4 | 3.838E+08 | 3.731E+07 | 2.492E+07 | 0.22 | < | 1.33 | 3.78 | 0 |

The chemiluminescent assay detects all oxidizing ROS including the protonated form of superoxide anion and hydrogen peroxide. Accordingly, the results obtained strongly suggest that DHQ, in addition to its well-known antioxidant property, inhibits the NADPH oxidase activation in PMN triggered by either FMLP or PMA. This property has been confirmed by the fact that DHQ strongly inhibits the oxygen consumption of PMN triggered by PMA, FMLP and opsonized zymosan (data not shown).

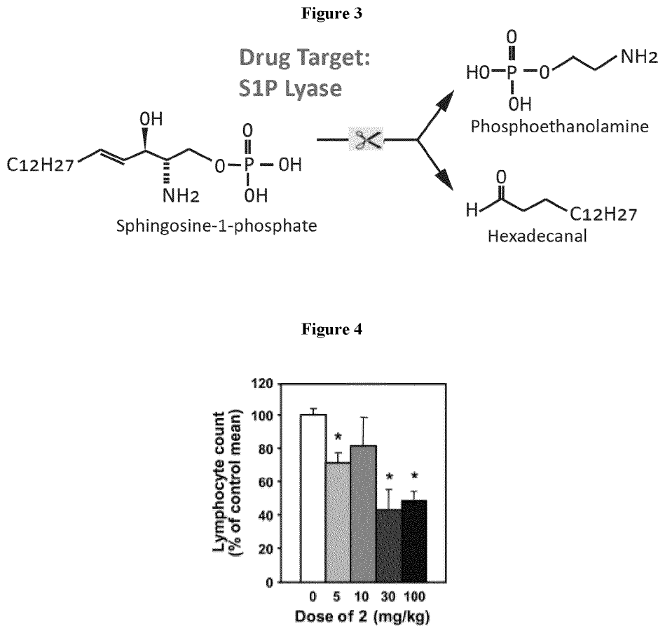

Effect of a S1P lyase inhibitor (S1PLI) on the circulating lymphocytes Sphingosine 1 phosphate (S1P) is one of the most abundant biologically active lysophospholipids in circulation. It is present in all mammalian cells and can serve as a second messenger in signal transduction pathways which regulate cell differentiation and apoptosis. S1P is also an agonist of five different G-protein coupled receptors, designated S1P1-S1P5. Autocrine and paracrine interactions between S1P and its receptors can modulate a wide range of physiological activities including angiogenesis, resistance to apoptosis and immune responses. Interaction of S1P with one of its receptors, S1P1, leads to inhibition of lymphocyte egress from primary and secondary lymphoid tissues, and results in depletion of recirculating lymphocytes from the peripheral blood. Enzymes of the S1P metabolic pathway may provide further intervention points for improved therapeutic applications. Systemic and local S1P levels are regulated directly by three enzyme classes. Sphingosine kinases phosphorylate sphingosine to produce S1P, which in turn is a substrate of S1P phosphatases. There are at least two routes of S1P metabolism: S P phosphatase (SPP) and S1P lyase. The major route of S1P degradation is via S1P lyase. S1P lyase catalyzes the irreversible cleavage of S1P at the C2-3 carbon bond giving rise to a long-chain aldehyde (2-hexadecanal) and phosphoethanolamine (see FIG. 3).

S1P lyase activity is found in all mammalian tissues except for platelets and erythrocytes. Although each of these enzyme classes are present in most mammalian cells, their relative abundance varies by tissue and cell type. In addition, cells have different capacities to discharge S1P stores into the extracellular environment and may respond differently to S1P generated within the cell. Over-expression of S1P lyase induces apoptosis in response to apoptotic stimuli, resulting in diminished intracellular S1P levels and increased levels of 2-hexadecanal and phosphoethanolamine, the former of which interacts with the proapoptotic protein BAX; and increases stress-induced responses. S1PL modulation impacts NF-kB and p38 signaling pathways which directly influences pro-inflammatory signals, including type I IFN production and IL-6 release.

The inhibition of S1P lyase results in elevated S1P levels in various body compartments and organs, including the thymus and lymph nodes. S1P upon ligation to its specific cell surface receptors can limit the production of IL12 and IL23 production but increase IL27 levels in dendritic cells activated with LPS, an effect previous demonstrated to regulate early innate immune responses after viral infection, and a desired phenotype to control coronavirus infection.

Increased S1P levels impair the generation of the S1P gradient which controls the release of T lymphocytes from the thymus.

The subsequent decrease in cellular influx from the circulation into the infected lungs reduces inflammation, and hence ameliorates the clinical symptoms of MERS-CoV and/or SARS-CoV.

The immune modulation achieved by treatment with compounds S1PLI is of therapeutic benefit in inflammatory diseases. Dose escalation studies indicated that single oral doses of 30-100 mg/kg2 administered to mice induced 40-60% lymphopenia, while lower doses have a minimal to non-statistically significant effect (FIG. 4).

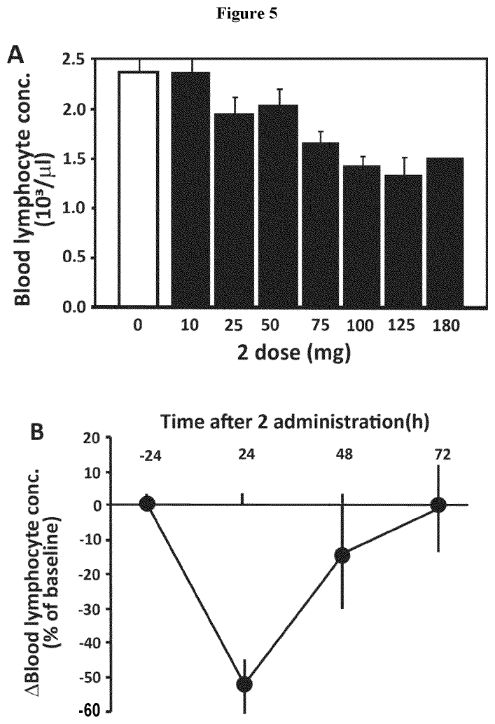

Along this line, phase 1 clinical trials were initiated to determine safety in human subjects. As a surrogate biomarker of S1PL inhibition, blood lymphocyte populations were determined by CBC analysis. As shown in FIG. 5A, a single ascending dose provided a clear dose responsive relationship, resulting in up to a ˜50% decrease in peripheral lymphocytes at the highest administered dose of 180 mg. The compound was generally well tolerated in all dose groups. Importantly, the induced reduction in circulating blood lymphocytes (N=6, 125 mg single dose) was reversible (FIG. 4B); lymphocyte populations rebounded to predose levels by 48 h after nadir.

### CONCLUSION

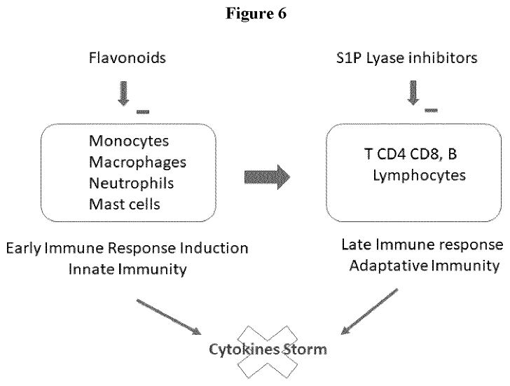

The concomitant inhibition of immune cell activation, especially that of neutrophils, and the decrease in lymphocyte and other immune cells into the lung of infected patients result in a strong decrease in inflammatory cytokines and chemokines in lung tissue and hence decrease the morbidity of patients infected by coronaviruses, such as MERS-CoV and SARS-CoV, including the COVID-19 strain (see FIG. 6).

1. A pharmaceutical combination of a flavonoid compound and a sphingosine-1-phosphate lyase inhibitor (S1PLI) for simultaneous, separate or sequential administration,
wherein the flavonoid compound is selected from the group consisting of dihydroquercetin (DHQ), quercetin, astilbin, dihydrokaempferol, butin, eriodictyol, hesperetin, hesperidin, homoeriodictyol, isosakuranetin, naringenin, naringin, pinocembrin, poncirin, sakuranetin, sakuranin, sterubin, epigallocatechin gallate, catechin and epicatechin, and
wherein the sphingosine-1-phosphate lyase inhibitor (S1PLI) is a compound of Formula I 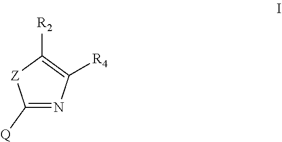
wherein
Z is selected from the group consisting of O, S, and NH;
Q is 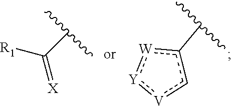
X is O or NR₃;
each of W, Y, and V is independently selected from the group consisting of CH₂, CH, N, NH, O and S;
R₁ is selected from the group consisting of OR_{A}, NHOH, hydrogen, optionally substituted alkyl, optionally substituted aryl, optionally substituted alkylaryl, optionally substituted arylalkyl, optionally substituted heteroalkyl, optionally substituted heterocycle, optionally substituted alkylheterocycle, and optionally substituted heterocyclealkyl;
R₂ is selected from the group consisting of OR_{B}, C(O)OR_{B}, hydrogen, halogen, nitrile, optionally substituted hydroxyalkyl, optionally substituted alkyl, optionally substituted aryl, optionally substituted alkylaryl, optionally substituted arylalkyl, optionally substituted heteroalkyl, optionally substituted heterocycle, optionally substituted alkylheterocycle, and optionally substituted heterocyclealkyl;
R₃ is selected from the group consisting of OR_{C}, N(R_{C})₂, NHC(O)R_{C}, NHSO₂R_{C}, and hydrogen;
R₄ is selected from the group consisting of OR_{D}, OC(O)R_{D}, N(R_{E})₂, hydrogen, halogen, optionally substituted hydroxyalkyl, optionally substituted alkyl, optionally substituted aryl, optionally substituted alkylaryl, optionally substituted arylalkyl, optionally substituted heteroalkyl, optionally substituted heterocycle, optionally substituted alkylheterocycle, and optionally substituted heterocyclealkyl;
each of R_{A}, R_{B}, R_{C}, R_{D}, and R_{E} is independently selected from the group consisting of hydrogen, optionally substituted alkyl, optionally substituted aryl, optionally substituted alkylaryl, optionally substituted arylalkyl, optionally substituted heteroalkyl, optionally substituted heterocycle, optionally substituted alkylheterocycle, and optionally substituted heterocyclealkyl.

2. The pharmaceutical combination of claim 1, wherein the flavonoid compound is selected from the group consisting of dihydroquercetin (DHQ), quercetin, astilbin, epigallocatechin gallate, catechin and epicatechin.

3. The pharmaceutical combination of claim 1, wherein
Q is

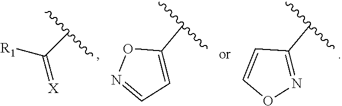

4. The pharmaceutical combination of claim 1, wherein R₁ is C₁-C₅ alkyl.

5. The pharmaceutical combination of claim 1, wherein R₂ is hydrogen or —(CH₂)ₙ—OH, wherein n is 1 to 5.

6. The pharmaceutical combination of claim 1, wherein R₄ is hydrogen, tetrahydroxybutyl, or —(CH₂)ₙ—OH, wherein n is 1 to 5.

7. The pharmaceutical combination of claim 1, wherein the sphingosine-1-phosphate lyase inhibitor (S1PLI) compound of Formula I is selected from the group consisting of

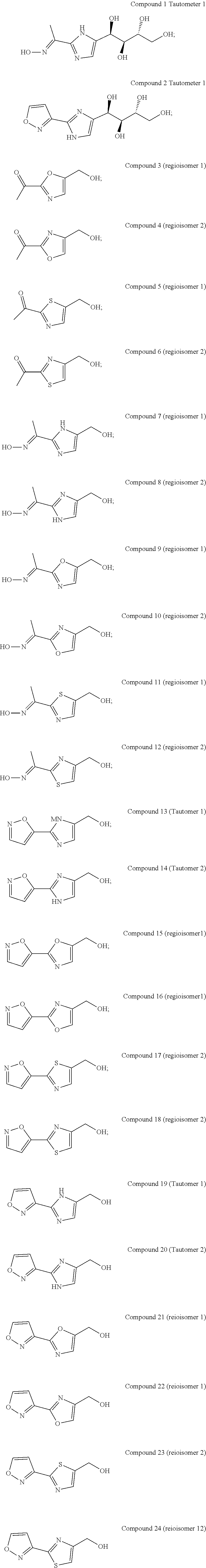

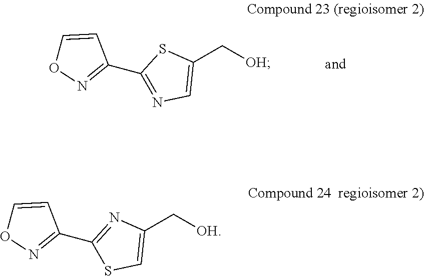 [SMILES: OCc1cnc(-c2ccon2)s1 ; OCc1csc(-c2ccon2)n1]

8. The pharmaceutical combination of claim 1, wherein the sphingosine-1-phosphate lyase inhibitor (S1PLI) compound of Formula I is

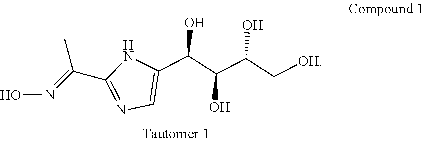 [SMILES: C/C(=N\O)c1ncc([C@@H](O)[C@H](O)[C@H](O)CO)[nH]1]

9. A method for ameliorating and/or reducing lung inflammation; resulting from MERS-CoV and/or SARS-CoV virus infection, during and following the virus infection of a subject, the method comprising administering a pharmaceutical combination of claim 1 to the subject.

10. A method for ameliorating and/or reducing lung inflammation resulting from MERS-CoV and/or SARS-CoV virus infection, during and following the virus infection of a subject, the method comprising co-administering to the subject in need thereof a therapeutically effective amount of a flavonoid compound and a sphingosine-1-phosphate lyase inhibitor.

11. The method of claim 10, wherein the sphingosine-1-phosphate lyase inhibitor is a compound of Formula I 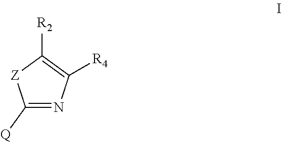
wherein
Z is selected from the group consisting of O, S, and NH;
Q is 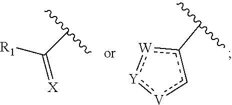
X is O or NR₃;
each of W, Y, and V is independently selected from the group consisting of CH₂, CH, N, NH, O and S;
R₁ is selected from the group consisting of OR_{A}, NHOH, hydrogen, optionally substituted alkyl, optionally substituted aryl, optionally substituted alkylaryl, optionally substituted arylalkyl, optionally substituted heteroalkyl, optionally substituted heterocycle, optionally substituted alkylheterocycle, and optionally substituted heterocyclealkyl;
R₂ is selected from the group consisting of OR_{B}, C(O)OR_{B}, hydrogen, halogen, nitrile, optionally substituted hydroxyalkyl, optionally substituted alkyl, optionally substituted aryl, optionally substituted alkylaryl, optionally substituted arylalkyl, optionally substituted heteroalkyl, optionally substituted heterocycle, optionally substituted alkylheterocycle, and optionally substituted heterocyclealkyl;
R₃ is selected from the group consisting of OR_{C}, N(R_{C})₂, NHC(O)R_{C}, NHSO₂R_{C}, and hydrogen;
R₄ is selected from the group consisting of OR_{D}, OC(O)R_{D}, N(R_{E})₂, hydrogen, halogen, optionally substituted hydroxyalkyl, optionally substituted alkyl, optionally substituted aryl, optionally substituted alkylaryl, optionally substituted arylalkyl, optionally substituted heteroalkyl, optionally substituted heterocycle, optionally substituted alkylheterocycle, and optionally substituted heterocyclealkyl;
each of R_{A}, R_{B}, R_{C}, R_{D}, and R_{E} is independently selected from the group consisting of hydrogen, optionally substituted alkyl, optionally substituted aryl, optionally substituted alkylaryl, optionally substituted arylalkyl, optionally substituted heteroalkyl, optionally substituted heterocycle, optionally substituted alkylheterocycle, and optionally substituted heterocyclealkyl.

12. The method of claim 10, wherein the flavonoid compound is selected from the group consisting of dihydroquercetin (DHQ), quercetin, astilbin, dihydrokaempferol, butin, eriodictyol, hesperetin, hesperidin, homoeriodictyol, isosakuranetin, naringenin, naringin, pinocembrin, poncirin, sakuranetin, sakuranin, sterubin, epigallocatechin gallate, catechin and epicatechin.
